(19) United States
(12) Patent Application Publication
(10) Pub. No.: US 2012/0095286 A1
(43) Pub. Date: Apr. 19, 2012

(54) CATHETER FOR AN EMBRYO TRANSFER AND A CARRIER FOR A CELL TRANSPORT

(76) Inventor: CEZARY GRYGORUK, Bialystok (PL)

(21) Appl. No.: 13/274,732

(22) Filed: Oct. 17, 2011

(30) Foreign Application Priority Data
Oct. 18, 2010 (PL) PL-392662

(51) Int. Cl. A61B 17/435

(52) U.S. Cl. 600/34

(57) ABSTRACT

A catheter for introduction, particularly of the embryos, comprising an aspirator combined with an inner part of the catheter, characterized in that the core of the catheter is placed (1) inside the inner part of the catheter (4) and the aspirator (2), and the carrier for a cell transport, in particular of the embryos is covered with the adhesive agent.

The embryo transfer is the last step of the infertility treatment process using in vitro fertilization technique. At present, the catheter combined with 1 ml insulin syringe is used for the embryo transfer. In most solutions, the catheter consists of two elements: an outer casing and an inner part. Before taking the embryo in the catheter, the outer casing is pulled towards an attendant, and an exposed distal end of the inner part is immersed in a liquid culture medium comprising the embryos designed for the transfer. Subsequently, by pulling off a piston of the syringe connected to a proximal end of the inner part of the catheter, a negative pressure is formed in the catheter, so that the culture medium together with the embryos is gathered inside the catheter. The culture medium together with the embryos takes about 10% of the length of the catheter inner part. Next, the end of the catheter inner part is removed from the culture medium and shielded with the outer part of the catheter by sliding it down along the inner part, until the catheter inner part is completely covered. Thus prepared catheter is introduced into a cervical canal. After the penetration of the catheter to a depth of about 2-4 cm, the inner part of the catheter is slid out from the outer casing and introduced very delicately into an uterine cavity. When the distal end of the catheter inner part reaches an appropriate position in the uterine cavity, an injection of the embryos into the uterine cavity is performed. The pressure necessary for pushing out the embryos from the catheter is generated by the syringe connected to the proximal section of the inner part of the catheter. Performed studies showed that the pressure of the transferred fluid during the injection of the embryos changes very rapidly and can reach a value of 155 mmHg in less than 0.1 s, which translates into pressure increase of about 72 000 mmHg/s and subsequent pressure drop of about 144 000 mmHg/s. Measured injection velocity of the embryos in the uterine cavity reached as much as 21 m/s (75 km/h). Such rapid changes of the physical parameters during the transfer can lead to damage of the transferred embryos, which was showed in the studies performed on the murine embryos. In the group of the embryos transferred by means of standard kit for the embryo transfer, i.e. 1 ml insulin syringe combined with the catheter, in 43% of blastocysts morphological changes (shrinkage and collapse) was observed, whereas the average apoptotic index was 52%. International patent application WO8200754 discloses the catheter for the embryos introduction having the inner part and a guide and the casing with holes 13. As mentioned above, a disclosed solution still has disadvantages such as required high pressure of the transferred fluid for pushing out of the embryos and thus high risk of damage of the transferred embryos. European patent application EP1049505 discloses the catheter additionally having the casing 32 for an opening 45 to release of the transferred embryos. Described catheter is still not devoid of the previously mentioned drawbacks, such as the risk of the damage of the transferred embryo at the moment of releasing it from the catheter. So there is still a need to provide the catheter for an embryo transfer, which at the moment of releasing it does not use high pressures that can lead to the embryo damages. Unexpectedly, the mentioned problem is solved by the present invention.

First subject of the invention is a catheter for introduction, particularly the embryos, comprising an aspirator combined with an inner part of the catheter characterized in that the catheter core is placed inside the inner part of the catheter and the casing of the aspirator. Preferably, the catheter according to the invention is characterized in that it comprises the outer casing of the catheter that is slid on the inner part of the catheter. Preferably, the catheter according to the invention is characterized in that the catheter core includes the holder, more preferably combined with a connector, with a carrier, to which a transfer platform is attached. Equally preferably, the catheter according to the invention is characterized in that the carrier to which the transfer platform is attached, is straight, more preferably bent by means of an articulated joint arranged at a distal end of the carrier. The use of the articulated joint allows optimal positioning of the transfer platform both inside the inner casing of the catheter and after sliding out of the inner casing of the catheter. Preferably, transfer platform according to the invention is characterized in that it comprises an adhesive agent for transferring of the embryos. Where the transfer platform does not include the adhesive agent, it preferably includes a surface in the shape of intestinal villi, of the “Gecko tape” type. Preferably, the catheter according to the invention is characterized in that the transfer platform is made of the compound selected from the group that includes polypropylene, polyamide, polyester, polyglycolic acid, and polyglactine. Equally preferably, the catheter according to the invention is characterized in that the holder includes the stabilizer at the distal end, preferably with gaps and it has a palm support at the proximal end. Preferably, the catheter according to the invention is characterized in that the aspirator includes the finger holder at the proximal end and a conical connector at the distal end, wherein a diameter of its working chamber is not larger than the diameter of the stabilizer. Preferably, the catheter according to the invention is characterized in that the transfer platform is detachable from the carrier and preferably it is made of non-toxic biocompatible and/or biodegradable materials selected from the group that includes polyglycolic acid, polyglactine, polysaccharide, e.g. agar, polypropylene, polyamide, polyester, polystyrene. Equally preferably, the catheter according to the invention is characterized in that the adhesive agent is a substance selected from the group that includes hydroxymethylcellulose, hyaluronic acid, hydroxyethylcellulose, methylcellulose, polyvinylpyrrolidone, guar gum, glucomannan, sodium alginate, thiols, silanes derivatives or other adhesive compound. The adhesive compound means a surface-active substance, which improves an adhesion of the embryos to the catheter core. Equally preferably, the catheter according to the invention is characterized in that the transfer platform is covered with antibodies specifically binding antigens of a transparent shell of the embryo. Equally preferably, the catheter according to the invention is characterized in that the transfer platform includes the rear holder to push by the carrier, and preferably it includes the front casing.

Second subject of the invention is a carrier for a cell transport, in particular for the embryos, characterized in that it is covered with the adhesive agent, preferably selected from the group that includes hydroxymethylcellulose, hyaluronic acid, hydroxyethylcellulose, methylcellulose, polyvinylpyrrolidone, guar gum, glucomannan, sodium alginate, thiols, silanes derivatives or other adhesive compounds. Preferably, the carrier according to the invention is characterized in that it is made of compound selected from the group that includes polypropylene, polyamide, polyester, polyglycolic acid, polyglactine. The carrier is characterized in that it has the form of the transfer platform connected separably or permanently with the guide of the medical device introduced into the cavity of the body. The medical device may be the catheter or the device of the other type used for a cell or chemical substance transport into the human body in order to leave it with or without a carrier.

The core of the catheter is constructed in such a way that it can be placed inside the presently used catheters for the embryo and cell transfer. The core of the catheter can consist of three basic parts: the holder, the connector and the carrier with the transfer platform, with or without the articulated joint. The core of the catheter can also consist of two basic parts: the holder and the carrier with the transfer platform, with or without articulated joint. Additionally, the aspirator is used for the connection of the core of the catheter with the catheter. The maximal diameter of the carrier with the transfer platform is less at least by 0.05 mm from the inner diameter of the inner part of the catheter. It is important that the distance between the walls of the carrier and the walls of the catheter is large enough to allow unrestricted flow of the liquid without creating the conditions to a local increase in the pressure in the fluid surrounding the transfer platform with the embryos. The length of the carrier usually corresponds to the length of a flexible part of the inner part of the catheter, and it is usually about 7 cm, but in the case where is no connector used, the length of the carrier will be equal or larger than the length of the catheter inner part. The transfer platform is placed at the distal end of the carrier. It is the most forward part of the catheter core. The carrier can be directly or indirectly connected with the transfer platform by means of the articulated joint. The long axis of the articulated joint is situated at an angle of ≧90° in relation to the long axis of the carrier and at an angle of ≧90° in relation to the long axis of the transfer platform. The articulated joint allows the optimal placement of the transfer platform both inside the inner casing of the catheter and after it exits the inner casing of the catheter. The minimal length of the transfer platform is equal to the diameter of the transferred embryo, cell. The transfer platform may be permanently connected with the carrier and oriented along the long axis of the carrier or it may be the surface that is perpendicular to the long axis of the carrier. The transfer platform can also be an independent element and it can be separated from the carrier. In such case, the transfer platform remains in the uterine cavity together with the embryos, for this reason biocompatible and/or biodegradable material selected from the group that includes polyglycolic acid, polyglactine, polysaccharide, e.g. agar, polypropylene, polyamide, polyester, polystyrene should be used for its construction. In the case where the transfer platform is permanently attached to the carrier, the following materials can be used: polypropylene, polyamide, polyester and biodegradable polyglycolic acid, polyglactine. In the present invention, the embryos are temporarily attached to the transfer platform of the catheter core. The main aim of the attachment of the embryos to the transfer platform of the catheter is maintenance of the embryos in the longitudinal axis of the catheter away from the walls of the catheter during the entire process of transfer, so that an impact of shearing forces appearing during the liquid flow in the catheter is minimized. The temporary attachment of the embryos to the transfer platform can be done by means of the following compounds: hydroxymethylcellulose, hyaluronic acid, hydroxyethylcellulose, methylcellulose, polyvinylpyrrolidone, guar gum, glucomannan, sodium alginate or other adhesive compounds. These compounds demonstrate considerable plasticity and viscosity, so that they easily adhere both to the transfer platform and tissues. And the affinity to the tissues is much higher due to the larger roughness of the tissue surface in relation to materials, of which the transfer platform is made. The above properties favor the embryos placing in the uterine cavity. Transfer platform can also have a specific structure of the intestinal villi type, e.g. the material of Gecko Tape type or other structure that increases the surface tension. In order to fixing the embryo, it can also be covered with antibodies specifically binding antigens of the transparent shell of the embryo. The holder allows abutment and stabilization of the core of the catheter against a palm part of the hand during the transfer. The palm holder includes the stabilizer, made of a synthetic gum without content of latex, which provides an adequate shift resistance and the stabilization of the aspirator shift. The outer diameter of the palm holder stabilizer corresponds to the inner diameter of the working chamber of the aspirator. Moreover, the stabilizer of the palm holder includes gaps for the air pressure compensation on both sides of the stabilizer, so that inside the catheter is not produced pressure that could push out the fluid with the embryos from the inner part of the catheter. The palm holder includes a palm support with the diameter of at least 15 mm. The palm holder can be made of polypropylene, polyamide, and polyester. A connecting member is designed to stably connect the holder with a carrier member of the catheter core. Its alternative or improvement in the form of articulated joint allows optimal positioning of the transfer platform both inside the inner casing of the catheter and after sliding out of the inner casing of the catheter. The connection should be rigid enough, so that the shift of the palm stabilizer results in equal shift of the carrier member inside the catheter, and simultaneously it should be flexible enough, so that not to restrict plasticity of the catheter. For example, stainless steel wire with the diameter of 0.1 mm±0.02 meets such conditions. The length of the connecting member depends on the length of the used catheter and is chosen so that the fluid being drawn into the catheter has no contact with the connecting member. The connection between the connector and the holder and the carrier member is thermally formed. The aspirator allows pulling of the catheter from above the inner part of the transfer platform and the carrier part of the catheter core. For example, the aspirator of the catheter according to the invention is characterized in that a flange has a diameter of at least 15 mm. The working chamber of the aspirator has the inner diameter that corresponds to the diameter of the palm holder stabilizer, and the length of the working chamber is at least 30 mm. The aspirator is connected with the flange of the inner part of the catheter by means of conical connector that is providing stable connection. The aspirator can be made of polypropylene or polyethylene terephthalate. During the transfer, after placing the catheter in appropriate position in the uterine cavity, an operator abutting his palm against the holder of the core of the catheter pulls the aspirator with palm forefinger, so that the inner part of the catheter is retracted from above the transfer platform and the carrier part of the catheter core. The operator has a direct impact on the speed of the inner casing shift of the catheter. By using the core of the catheter it is not necessary to produce the pressure to place the embryos inside the uterine cavity. The embryos are placed in the uterine cavity by using the carrier with the transfer platform. The catheter serves only to secure, autraumatic moving of the core of the inner catheter through the cervical canal.

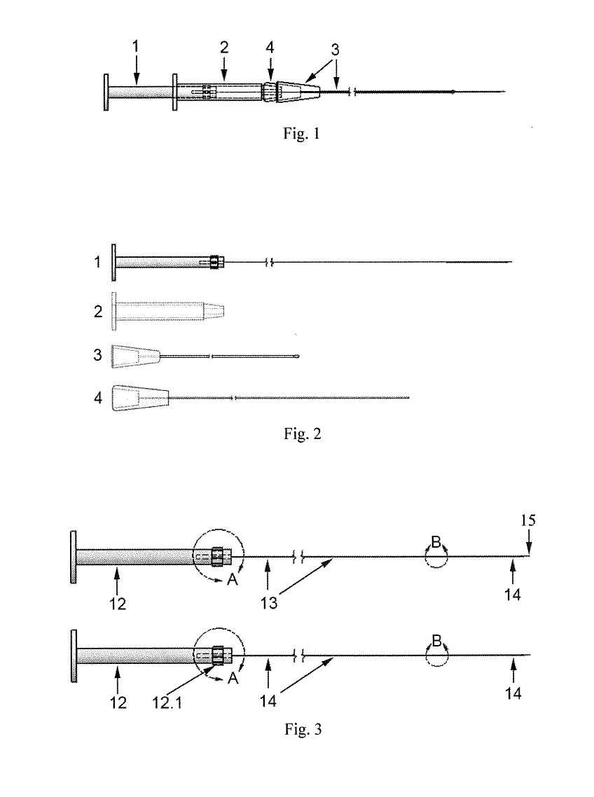

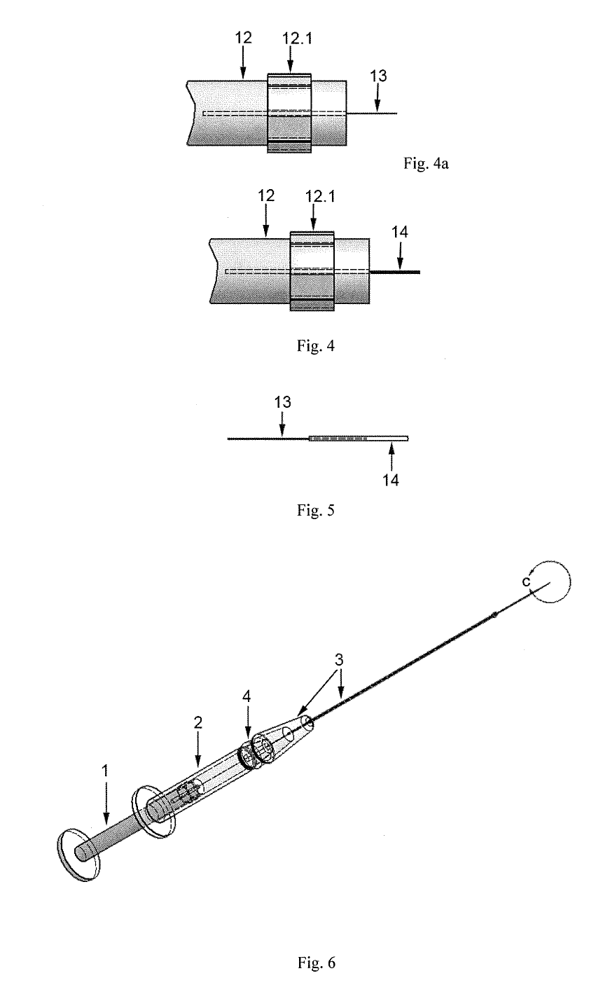

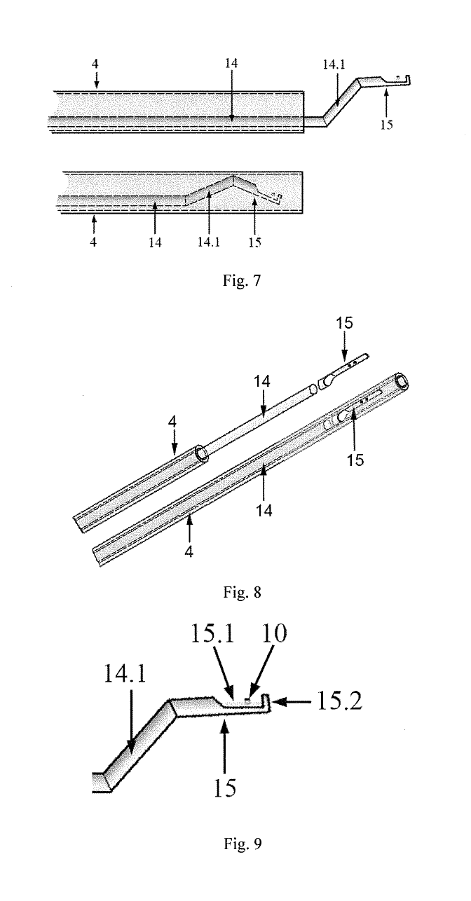

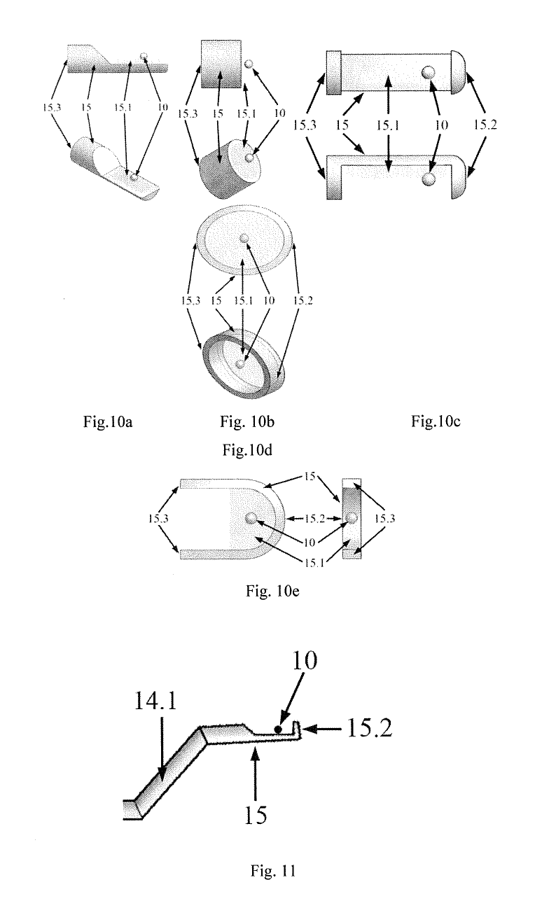

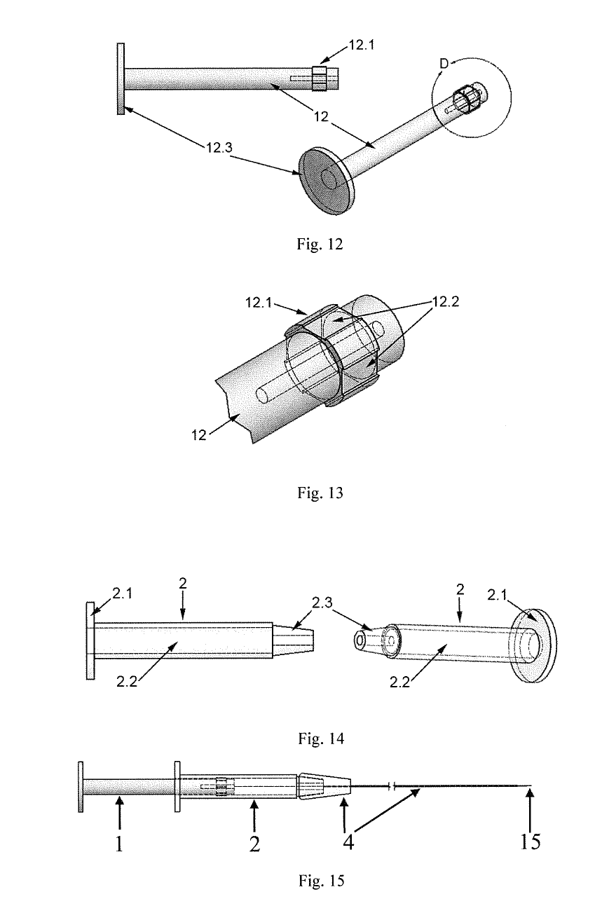

The examples of the embodiments of the invention are presented in the accompanying drawings, where: FIG. 1 shows a view of completed kit for an embryo transfer consisting of: the core of the inner catheter 1, the aspirator 2, the inner part of the catheter 4, the outer casing of the catheter 3, FIG. 2 shows separately individual elements of the kit for the embryo transfer consisting of: the core of the inner catheter 1, the aspirator 2, the inner part of the catheter 4, the outer casing of the catheter 3, FIG. 3 shows the core of the catheter consisting of: the holder 12, the connector 13 of the carrier 14 and the transfer platform 15, FIG. 4 shows how to connect the holder 12 with the connector 13 (FIG. 4a) or directly with the carrier 14 (FIG. 4), FIG. 5 shows how to connect the connector 13 with the carrier 14, FIG. 6 shows a view of completed kit for the embryo transfer consisting of: the core of the inner catheter 1, the aspirator 2, the inner part of the catheter 4, the outer casing of the catheter 3, FIG. 7 shows the transfer platform 15 attached permanently to the carrier 14 with exposed and pulled over inner part of the catheter 4 with the articulated joint 14.1, FIG. 8 shows the transfer platform 15 in the embodiment separated from the carrier with exposed and pulled over inner part of the catheter 4, FIG. 9 shows the transfer platform 15 attached to the carrier 14, FIG. 10a, b, c, d, and e show the transfer platform 15 that is independent of the carrier with the rear holder 15.3 and the front casing 15.2, FIG. 11 shows the transfer platform 15 permanently attached to the carrier with the embryos temporarily attached without the use of additional substances, FIG. 12 shows the palm holder of the catheter 12, FIG. 13 shows the stabilizer 12.1 with holes of the palm holder of the catheter, FIG. 14 shows the aspirator, and FIG. 15 shows the catheter according to the invention without the outer casing of the catheter 3.

### EXAMPLE 1

The core of the catheter 1 consists of the carrier 14 made of polypropylene, that has the length of 100 mm and the diameter of 0.25 mm. At the distal end of the carrier 14 is formed the transfer platform 15 that has the length of 2 mm, a width of 0.2 mm and a height of 0.08 mm and is rounded at the end. The section of 1 mm of the transfer platform 15 is covered by thin <0.05 mm layer of 1% aqueous solution of hydroxymethylcellulose 15.1, which is left to dry. At its proximal end the carrier 14 is thermally attached to the connector 13 of the core of the catheter 1 made of stainless steel that has the length of 210 mm and the diameter of 0.1 mm. At its proximal end the connector 13 is thermally attached to the holder 12 made of polypropylene. The holder 12 has the length of 85 mm, the diameter of 4.9 mm, and at the proximal end it includes the palm support 12.3 with the diameter of 20 mm and the thickness of 2 mm. Moreover, the holder 12 includes the stabilizer 12.1 with the diameter of 5 mm and the length of 5 mm with four symmetrically arranged gaps 12.2, each with a dimension of 1 mm, on the perimeter of the stabilizer 12.1. The stabilizer 12.1 is made of gum and is located 3 mm from the distal end of the holder 12.3. On the holder 12 of the core of the catheter 1 is slid over the aspirator 2 of a cylindrical shape, that is made of polypropylene with the outer diameter of 7 mm, the length of 75 mm, with the working chamber 2.2 with the diameter of 5 mm and the length of 75 mm, ended at the proximal end with the finger holder 2.1 having the diameter of 20 mm and the thickness of 2 mm made of polypropylene and ended at the distal end with the conical connector 2.3 made of polypropylene having the length of 8 mm and the shape adjusted to the shape of the flange of the inner part of the catheter. The core of the catheter 1 is inserted inside the catheter to produce stable connection between the aspirator 2 and the inner part of the catheter 4. The degree of shield of the transfer platform 15 by the distal end of the inner part of the catheter 4 can be adjusted by means of aspirator 2. Hydration of hydroxymethylcellulose occurs after exposing the transfer platform 15 and placing it in the medium comprising the embryos 10 for a transfer. Next, the embryos 10 are deposited on the transfer platform 15 on the surface of hydrated hydroxymethylcellulose. Such prepared transfer platform is slid over by the inner part of the catheter 4 by means of the aspirator 2, and then is slid over by the outer casing of the catheter 4. During this process important is that the embryos 10 still remain in the liquid medium of the culture medium. The catheter together with the embryos 10 deposited on the transfer platform 15 of the catheter in the environment of the culture medium are transferred into the uterine cavity, where, by means of aspirator 2 of the casing, the inner casing of the catheter 4 is slid down to expose the transfer platform 15 with the embryos 10. By delicate withdrawing the core of the catheter 1, the embryos 10 are left in the uterine cavity. The embryos 10 are placed in the uterine cavity to prevent damage of the embryos during the transfer. The catheter according to the example is illustrated in FIG. 1, FIG. 2, FIG. 3, FIG. 4, FIG. 5, FIG. 6, FIG. 12, FIG. 13, FIG. 14 and FIG. 15.

### EXAMPLE 2

The catheter as in the example 1, with the difference that the transfer platform 15 is detachable from the carrier 14. The detachable transfer platform according to the example 2 is shown in FIG. 8.

### EXAMPLE 3

The catheter as in the example 1 with the difference that the embryos 10 are attached to the transfer platform 15 without using the adhesive agent 15.1 by means of surface tension, as it is shown in FIG. 11.

### EXAMPLE 4

The catheter as in the example 1 with the difference that it does not include the outer casing of the catheter 3. The embryos 10 are placed in the uterine cavity by pulling off the inner part of the catheter 4 from above the transfer platform 15 of the core of the catheter 1. By withdrawing the core of the catheter 1 together with the inner part of the catheter 4, the embryos 10 are left in the uterine cavity. Catheter without the outer casing 3 with the transfer platform 15 is shown in FIG. 15

### EXAMPLE 5

The catheter as in the example 1 with the difference that the carrier is connected directly with the holder without the mediation of the connector 13, and it is connected to the transfer platform by means of articulated joint 14.1, as it is shown in FIG. 7, FIG. 9, FIG. 3 (new added figure), FIG. 4 (new added figure).

### EXAMPLE 6

The catheter as in the example 6 with the difference that the embryos are attached to the transfer platform 15 without using additional substances, as it is shown in FIG. 11.

1. A catheter for introduction, particularly of the embryos, comprising an aspirator combined with an inner part of the catheter, characterized in that the catheter core (1) is placed inside the inner part of the catheter (4) and the aspirator (2).

2. The catheter according to claim 1, characterized in that it comprises an outer casing of the catheter (3) that is slid on the inner part of the catheter (4).

3. The catheter according to claim 1, characterized in that the core of the catheter (1) includes a holder (12), preferably combined with a connector (13), with a carrier (14), to which a transfer platform is attached (15), preferably with the adhesive agent (15.1) for transferring of the embryos.

4. The catheter according to claim 1, characterized in that the carrier (14) to which the transfer platform is attached (15) is straight, preferably bent by means of an articulated joint (14.1) arranged at a distal end of the carrier (14)

5. The catheter according to claim 1, characterized in that the transfer platform (15) is made of the compound selected from the group that includes polypropylene, polyamide, polyester, polyglycolic acid, polyglactine.

6. The catheter according to claim 1, characterized in that the holder (12) includes the stabilizer at the distal end (12.1), preferably with gaps (12.2) and it has a palm support at the proximal end (12.3).

7. The catheter according to claim 1, characterized in that the aspirator (2) includes the finger holder at the proximal end (2.1) and a conical connector at the distal end (2.3), wherein a diameter of its working chamber (2.2) is not larger than the diameter of the stabilizer (12.1).

8. The catheter according to claim 1, characterized in that the transfer platform (15) is detachable from the carrier (14) and preferably is made of non-toxic biocompatible and/or biodegradable materials selected from the group that includes polyglycolic acid, polyglactine, polysaccharide, e.g. agar, polypropylene, polyamide, polyester, polystyrene.

9. The catheter according to claim 1, characterized in that the transfer platform (15) includes a rear holder (15.3) to push by the carrier, and preferably includes a front casing (15.2).

10. The catheter according to claim 1, characterized in that the adhesive agent is a substance selected from the group that includes hydroxymethylcellulose, hyaluronic acid, hydroxyethylcellulose, methylcellulose, polyvinylpyrrolidone, guar gum, glucomannan, sodium alginate, thiols, silanes derivatives or other adhesive compound.

11. The carrier for a cell transport, in particular of the embryos, characterized in that it is covered by the adhesive agent, preferably selected from the group that includes hydroxymethylcellulose, hyaluronic acid, hydroxyethylcellulose, methylcellulose, polyvinylpyrrolidone, guar gum, glucomannan, sodium alginate, thiols, silanes derivatives or other adhesive compound.

12. The carrier according to claim 11, characterized in that it is made of a compound selected from the group that includes polypropylene, polyamide, polyester, polyglycolic acid, polyglactine.

13. The carrier according to claim 11, characterized in that it has the form of the transfer platform (15) connected separably or permanently with the guide of the medical device introduced into the cavity of the body.
